(19) United States
(12) Patent Application Publication
(10) Pub. No.: US 2023/0255990 A1
(43) Pub. Date: Aug. 17, 2023

(54) USE OF 2'-FL FOR TREATMENT OF SYMPTOMS ASSOCIATED WITH AUTISM SPECTRUM DISORDER

(71) Applicant: Glycosyn LLC, Waltham, MA (US)

(72) Inventors: Ardythe L. Morrow, Cincinnati, OH (US); Jeffrey Alberts, Bloomington, IN (US)

(21) Appl. No.: 18/015,127

(22) PCT Filed: Jul. 8, 2021
(86) PCT No.: PCT/US2021/040915

§ 371 Date: Jan. 9, 2023

Related U.S. Application Data
(60) Provisional application No. 63/049,503, filed on Jul. 8, 2020.

(51) Int. Cl. A61K 31/702; A61P 25/00

(52) U.S. Cl. CPC A61K 31/702; A61P 25/00

(57) ABSTRACT

Provided herein are compositions and methods for reducing a symptom of autism spectrum disorder in a subject such as a human infant or child. The method involves administering to the subject a composition comprising purified 2′-fucosyllactose.

### CROSS-REFERENCE TO RELATED APPLICATIONS

This application claims the benefit under 35 U.S.C. § 119(e) of U.S. provisional application No. 63/049,503, filed Jul. 8, 2020, the contents of which are incorporated by reference herein in their entirety.

### BACKGROUND OF THE INVENTION

More than 1% of the population world-wide may be affected by Autism Spectrum Disorder (ASD), which has a major life-long impact. Individuals with ASD characteristically have persistent deficits in social communication and interaction and restricted, repetitive patterns of behavior. Such behaviors are occur early in life.

### SUMMARY OF THE INVENTION

The invention provides a solution to the pressing problem of treating individuals with ASD. The invention features a method for reducing a symptom of ASD in a subject by administering to the subject a composition comprising purified 2′-fucosyllactose. The subject is characterized or identified as comprising a symptom of ASD. Criteria for diagnosing or identifying an individual characterized as comprising ASD is known in the art and described herein. Preferably, the subject is a human. For example, the subject is less than 7 years of age, e.g., less than 3 months of age. The methods are also used to treat older children, teenagers, and even adults with one or more symptoms of ASD. For example the symptom comprises one or more deficits in socialization and/or cognition. Other exemplary symptoms include gastrointestinal dysfunction, microbial disbiosis, mitochondrial dysfunction, thermal dysregulation, or immune dysregulation.

The fucosylated oligosaccharide, e.g., 2′-FL, is purified for use in therapeutic or nutritional products. 2′-FL is administered orally using an edible composition or a drinkable composition. For example, the the composition comprises a food product, e.g., a pureed food product. In another example, the composition in the form of an infant formula, e.g., a liquid or powder formula product. Also within the invention is a composition comprising purified 2′-FL for use in a treatment to reduce reducing a symptom of autism spectrum disorder in a subject.

A purified fucosylated oligosaccharide produced by the methods described above is also within the invention. A purified oligosaccharide, e.g., 2′-FL, is one that is at least 90%, 95%, 98%, 99%, or 100% (w/w) of the desired oligosaccharide by weight. Purity is assessed by any known method, e.g., thin layer chromatography or other electrophoretic or chromatographic techniques known in the art. The invention includes a method of purifying a fucosylated oligosaccharide produced by a genetically engineered bacterium that produces 2′-FL, which method comprises separating the desired fucosylated oligosaccharide (e.g., 2′-FL) from contaminants in a bacterial cell extract or lysate, or bacterial cell culture supernatant. Contaminants include bacterial DNA, protein and cell wall components, and yellow/brown sugar caramels sometimes formed in spontaneous chemical reactions in the culture medium.

By the terms “effective amount” and “therapeutically effective amount” of a formulation or formulation component is meant a nontoxic but sufficient amount of the formulation or component to provide the desired effect.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a bar graph showing the marginal mean of aggregons including dams in a mouse model of ASD.

FIG. 2 is a diagram showing scoring of contact behavior. Each pup in the litter was scored each minute for how many males (blue pups) and females (pink pups) they were in contact with, excluding contacts via tails and outstretched paws. Each combination of contacts was assigned a unique identifier or “contactogon”. For example, 0 M 2 F designates contact with zero males and two females. Contactogons possible for only a single sex (e.g., 3 M 0 F) are not shown, and were collapsed into a single category for the purposes of statistical analysis.

### DETAILED DESCRIPTION

The etiology of ASD is not known, but a number of co-morbidities frequently occur in individuals with ASD, including gastrointestinal symptoms, microbial dysbiosis, mitochondrial dysfunction, and immune dysregulation. Increased co-morbidities has been linked to increased severity of ASD behaviors. Oral treatment with 2′-FL alone or mixed with other human milk oligosaccharides (HMOs) are useful to address these co-morbidities and reduce symptoms of autism.

### Diagnosis of Autism Spectrum Disorder (ASD)

Diagnosing autism spectrum disorder (ASD) can be difficult, as there is no definitive medical test, e.g., a blood test, to diagnose the disorders. Doctors review a child's behavior and development to make a diagnosis.

ASD can be detected at 18 months or younger. By age 2, a diagnosis by an experienced professional can be considered very reliable (Lord et al., External Autism from 2 to 9 years of age. Arch Gen Psychiatry. 2006 June; 63(6):694-701). However, many children do not receive a final diagnosis until much older. This delay means that children with an ASD might not get the help they need.

Diagnosing an ASD involves two steps: (1) developmental screening, and (2) a comprehensive diagnostic evaluation.

Developmental screening is a short test to determine whether children are learning basic skills when they should, or if they might have delays. During developmental screening, the doctor might ask the parent some questions or talk and play with the child during an exam to see how she learns, speaks, behaves, and moves. A delay in any of these areas could be a sign of a problem. Children are screened for developmental delays and disabilities during regular well-child doctor visits at 9 months, 18 months, and at 24 or 30 months. Additional screening might be needed if a child is at high risk for developmental problems due to preterm birth, low birth weight or other reasons. Additional screening might be needed if a child is at high risk for ASD (e.g., having a sister, brother or other family member with an ASD) or if behaviors sometimes associated with ASD are present. All children should be screened for developmental delays, but especially to monitor those who are at a higher risk for developmental problems due to preterm birth, low birth weight, or having a brother or sister with an ASD. If s doctor observes signs of a problem, a comprehensive diagnostic evaluation is needed.

The second step of ASD diagnosis is a comprehensive evaluation. This thorough review may include observing the child's behavior and development and interviewing the parents. It may also include a hearing and vision screening, genetic testing, neurological testing, and other medical testing. In some cases, the primary care doctor might choose to refer the child and family to a specialist for further assessment and diagnosis. Specialists who can do this type of evaluation include developmental pediatricians, child neurologists, as well as child psychologists or psychiatrists.

Children who have been diagnosed with ASD or who are at risk of developing the disorder as treated with 2′-FL as described herein.

### Gastrointestinal Symptoms, Microbial Dysbiosis

Many children with ASD suffer from chronic gastrointestinal (GI) symptoms [Molloy C A, Manning-Courtney P. Prevalence of chronic gastrointestinal symptoms in children with autism and autism spectrum disorders. Autism 2003 June; 7(2):165-71; Li Q, Han Y, Dy A B C, Hagerman R J. The gut microbiota and autism spectrum disorders. Front Cell Neurosci. 2017; 11:1-14]. Several studies have correlated GI symptoms with severity of autism [Adams J B, Johansen L J, Powell L D, Quig D, Rubin A R. Gastrointestinal flora and gastrointestinal status in children with autism-comparisons to typical children and correlation with autism severity. BMC Gastroenterol. 2011; 11:1-13; Tomova A, Husarova V, Lakatosova S, Bakos J, Vlkova B, Babinska K, Ostatnikova D. Gastrointestinal microbiota in children with autism in Slovakia. Physiol Behav. 2015; 138:179-87]. Identifying the mechanism to explain these reports has been elusive; one study compared pathology in autistic and non-autistic children with GI symptoms but found no evident differences [Kushak R I, Buie T M, Murray K F, Newburg D S, Chen C, Nestoridi E, Winter H S. Evaluation of intestinal function in children with autism and gastrointestinal symptoms. J Pediatr Gastroenterol Nutr. 2016 May; 62(5):687-91. doi: 10.1097/MPG.0000000000001174. PMID: 26913756].

However, there is growing evidence that the gut microbiota has a role in ASD. Several studies of rats and mice reared in germ-free (GF) environments have shown that GF rodents are impaired in terms of social exploration or sociability and anxiety-related behavior (Desbonnet L, Clarke G, Shanahan F, Dinan T G, Cryan J F. Microbiota is essential for social development in the mouse. Mol Psychiatry. 2014 February; 19(2):146-8. doi: 10.1038/mp.2013.65; Arentsen T, Raith H, Qian Y, Forssberg H, Diaz Heijtz R. Host microbiota modulates development of social preference in mice. Microb Ecol Health Dis. 2015 Dec. 15; 26:29719. doi: 10.3402/mehd.v26.29719]. Multiple studies have reported significantly altered gut microbiota in ASD compared with healthy control children. The most consistent difference appears to be a lower abundance of bifidobacteria in children with autism compared to healthy control children Adams, 2011; Li, 2017; Tomova, 2015; Coretti L et al. Gut microbiota features in young children with autism spectrum disorder. Front Microbiol. 2018; 9: 3146. doi: 10.3389/fmicb.2018.03146. PMCID: PMC6305749. PMID: 30619212].

### Mitochondrial Dysfunction

Defects in mitochondrial oxidative phosphorylation have been reported by multiple studies of patients with ASD. In a large genetic study of mitochondrial DNA, significant differences in the distribution of mitochondrial haplotypes between ASD and control patients [Chalkia D, Singh L N, Leipzig J, Lvova M, Derbeneva O, Lakatos A, Hadley D, Hakonarson H, Wallace D C. Association between mitochondrial DNA haplogroup variation and autism spectrum disorders. JAMA Psychiatry. 2017 Nov. 1; 74(11):1161-1168. doi: 10.1001/jamapsychiatry.2017.2604]. In another study, researchers compared mitochondrial function in the gut mucosa of children with and without ASD [Rose S, Bennuri S C, Murray K F, Buie T, Winter H, Frye R E. Mitochondrial dysfunction in the gastrointestinal mucosa of children with autism: A blinded case-control study. PLoS One. 2017 Oct. 13; 12(10):e0186377. doi: 10.1371/journal.pone.0186377]. Mitochondrial function in cecal tissue from children with ASD differed significantly from control children who manifested similar GI symptoms but were neurotypical or who had Crohn's Disease. Thus, there is significant evidence implicating mitochondrial function as an important component of autism.

Mitochondrial deficits that impact gut function may be alleviated by 2′-FL administration. In a murine model, administration of 2′-FL to increase gastrointestinal mitochondrial function via pathways related to the electron transport chain, oxidative phosphorylation, and protein targeting to the mitochondrion Mezoff E A, Hawkins J A, Ollberding N J, Karns R, Morrow A L, Helmrath M A. The human milk oligosaccharide 2′-fucosyllactose augments the adaptive response to extensive intestinal resection. Am J Physiol Gastrointest Liver Physiol. 2016; 310(6):G427-38. Epub 2015/12/25. doi: 10.1152/ajpgi.00305.2015. PubMed PMID: 26702137; PMCID: PMCPMC4796291].

Thermal dysregulation

Autistic behavior is commonly observed to normalize during infection-associated fever [Good P. Simplifying study of fever's dramatic relief of autistic behavior. Clin Nutr ESPEN. 2017 February; 17:1-7. doi: 10.1016/j.clnesp.2016.09.002], suggesting that thermal dysregulation contributes to ASD. Bacterial colonization, or its depletion due to antibiotics, affects metabolism and thermoregulation.

Antibiotics were administered to mouse mothers in the perinatal period to determine the impact of microbial depletion on the offspring. Offspring microbiota, social behavior, response to maternal separation, and thermoregulatory functioning were then examined. The data indicated that antibiotic-exposed dams and their offspring exhibited a number of subtle differences in behavior and physiology consistent with ASD. These differences included offspring that were significantly cooler following maternal separation and huddling early in development. By enhancing mitochondrial bioenergetics, 2′-FL is useful to modify thermal regulation.

### Immune Dysregulation

Eftekharian et al. [Eftekharian M M et al. Cytokine profile in autistic patients. Cytokine. 2018 August; 108:120-126. doi: 10.1016/j.cyto.2018.03.034. https://doi.org/10.1016/j.cyto.2018.03.034] studied ASD patients and healthy controls and found that inflammatory cytokines TNF-α, IL-6 and IL-17 were up-regulated in ASD patients (P<0.001). In another study, cytokine production in peripheral blood mononuclear cells from 20 children with autism spectrum disorder were compared to matched control children [Molloy C A, Morrow A L, et al. Elevated cytokine levels in children with autism spectrum disorder. J Neuroimmunol. 2006 March; 172(1-2):198-205]. The results indicated that those with ASD had increased activation of both Th2 and Th1 arms of the adaptive immune response, with a Th2 predominance, but they lacked the expected compensatory increase in the regulatory cytokine IL-10. 2′-FL, administered alone or as part of an HMO mixture has been shown, in pre-clinical studies, to increase IL-10 and decrease inflammatory cytokine expression [Xiao L et al. Human milk oligosaccharides promote immune tolerance via direct interactions with human dendritic cells. Eur J Immunol. 2019 Mar. 22. doi: 10.1002/eji.201847971; Castillo-Courtade L. Attenuation of food allergy symptoms following treatment with human milk oligosaccharides in a mouse model. Allergy. 2015 September; 70(9):1091-102. doi: 10.1111/a11.12650; Comstock S S, Wang M, Hester S N, Li M, Donovan S M. Select human milk oligosaccharides directly modulate peripheral blood mononuclear cells isolated from 10-d-old pigs. Br J Nutr. 2014 Mar. 14; 111(5):819-28. doi: 10.1017/S0007114513003267; He Y, Liu S, Kling D E, Leone S, Lawlor N T, Huang Y, et al. The human milk oligosaccharide 2′-fucosyllactose modulates CD14 expression in human enterocytes, thereby attenuating LPS-induced inflammation. Gut. 2016; 65(1):33-46. Epub 2014/11/29. doi: 10.1136/gutjnl-2014-307544. PubMed PMID: 25431457; Yu Z T, Chen C, Kling D E, Liu B, McCoy J M, Merighi M, Heidtman M, Newburg D S. The principal fucosylated oligosaccharides of human milk exhibit prebiotic properties on cultured infant microbiota. Glycobiology. 2013 February; 23(2):169-77. doi: 10.1093/glycob/cws138. PMID: 23028202]. 2′-FL is useful to improve the cytokine profile in autism.

### 2′-FL as a Treatment for Autism-Associated Co-Morbidities and Behaviors

Alone or in combination with other HMOs, 2′-FL increases the growth and relative abundance of bifidobacteria [Yu, 2013; Elison E, Vigsnaes L K, Rindom Krogsgaard L, Rasmussen J, Sorensen N, McConnell B, et al. Oral supplementation of healthy adults with 2′-O-fucosyllactose and lacto-N-neotetraose is well tolerated and shifts the intestinal microbiota. Br J Nutr. 2016; 116(8):1356-68], which are typically lacking in ASD patients. 2′-FL is used to address deficits in mitochondrial functioning in ASD patients, and enhance thermoregulation, by enhancing mitochondrial energy processing. Further, 2′-FL enhances immune regulation, managing inflammatory response and enhancing IL-10 and T-regulatory cells. The gut microbiota and host immune status have been shown to influence neurobehavior. Thus, 2′-FL is useful as a treatment for symptoms and behaviors in autism.

Oral administration of 2′-FL as a dietary supplement at doses of approximately 1-100 g/day, e.g., 5 to 10 g/day, to reduce gastrointestinal symptoms, improve mitochondrial function, enhance immune health, and improve social behaviors in individuals with autism spectrum disorder (ASD).

### Therapeutic Compositions

2′-Fucosyllactose Powder (2′-FL) is a human milk oligosaccharide prebiotic. According to the FDA Guidance for Industry on Complementary and Alternative Medicine Products and Their Regulation by the Food and Drug Administration dated 2006, prebiotics are non-digestible food ingredients that affect the host beneficially by stimulating in a selective fashion the growth and/or activity of bacteria in the colon.

2′-Fucosyllactose (2′-FL) is a white homogenous powder and is neutral to slightly sweet with no off flavor. Dry matter makes up 96%, with a 4% moisture content. Results of analysis show that the overall content is: 2′-Fucosyllactose 93%, other sugars 3% and moisture 4%. For treatment, subjects take (or are administered) the 2′-FL each morning (e.g., at breakfast by adding the required amount to a drink or food) and optionally each afternoon/evening.

For example, administration of oligosaccharide is recommended for children of any age any time after diagnosis of ASD, to be continued as a routine daily dietary supplement. 2′-FL is administered at doses from 0.1 gram per day to 50 grams/day, e.g., 1-25 grams/day, 2.5-10 grams/day, e.g., 1, 2, 3, 4, 5, 6, 7, 8, 9 10 grams/day. An exemplary dosing regimen is 5 to 8 grams/day of 2′-FL alone or 2′-FL plus galacto-oligosaccharide (GOS), lacto-n-tetraose (LNT), lacto-n-neo-tetraose (LNneoT), inulin, or fructo-oligosaccharide, or other oligosaccharide. The oligosaccharide is a powder that can be provided including it in milk or other drink, or in cereal or other food (after cooking). Although doses for oligosaccharide is not typically administered as weight dependent or administered per unit weight of the subject, an approximate dose per kg body weight is in the range of 0.08 g to 0.8 g oligosaccharide/kg body weight.

It is recommended to divide the daily amount into approximately equal amounts given in the morning and the evening, with food or drink; thus, the oligosaccharide may be administered more than once per day, e.g. twice or thrice per day. Treatment outcomes include improved stooling (to improve frequency), improved microbial colonization (measured by 16S rDNA, PCR or whole genome sequencing to increase Bifidobacteria or other microbes capable of producing short chain fatty acid metabolites), reduced gastrointestinal symptoms, improved immune markers (increase in serum IL-10 as a regulatory immune marker, decreased allergy or decreased inflammatory cytokines; Molloy C A, Morrow A L, et al. Elevated cytokine levels in children with autism spectrum disorder. J Neuroimmunol. 2006 March; 172(1-2):198-205), increased social behaviors and decreased anti-social behaviors. Improvement in social and communication behaviors (e.g., interpersonal sociality) and decreased anti-social and repetitive behaviors are assessed using methods known in the art, e.g., Sparrow, S. S., Balla, D. A., & Cicchetti, D. V. (1984) Vineland Adaptive Behavior Scales, Classroom Edition. Circle Pines, Minn.: American Guidance Service and Bodfish, J. W., Symons, F. J. & Lewis, M. H. (1998). The Repetitive Behavior Scale: A test manual. Morganton: Western Carolina Center Research Reports. For example, interpersonal sociality is increase by at least 10%, e.g., 20%, 25%, 50%, 75%, 2-fold, 5-fold, 10-fold or more compared to the level of interpersonal sociality detected in the absence of 2′-FL treatment.

The composition may be formulated in different configurations. For example, the composition is processed into a solid configuration, e.g., as a powder. When present as a powder, the dimensions of the particles making up the powder may vary, ranging in some instances from 0.1 to 1000 microns, such as 1 to 500 microns.

Also provided are physiological acceptable compositions that include 2′-FL and a physiologically acceptable delivery vehicle. The compositions can be incorporated into a variety of formulations for administration to a subject. More particularly, the compositions can be formulated into physiological acceptable compositions by combination with appropriate, physiologically acceptable carriers or diluents, and may be formulated into preparations in solid, semi-solid, liquid or gaseous forms, such as tablets, capsules, powders, granules, ointments, solutions, suppositories, injections, inhalants and aerosols and topical compositions. The formulations may be designed for administration via a number of different routes, including oral, buccal, sublingual, rectal, parenteral, intraperitoneal, intradermal, transdermal, intracheal, etc., administration.

The physiological compositions may be in a form suitable for oral use, for example, as foods, tablets, troches, lozenges, aqueous or oily suspensions, dispersible powders or granules, emulsions, hard or soft capsules, or syrups or elixirs, gums, etc. Compositions intended for oral use may be prepared according to any convenient protocol for the manufacture of pharmaceutical compositions and such compositions may contain one or more agents selected from the group consisting of sweetening agents, flavoring agents, coloring agents and preserving agents.

In certain embodiments the composition is a food product. Food products of interest include the composition in combination with a food delivery vehicle. By food delivery vehicle is meant a delivery vehicle that is a nourishing substance that is eaten, drunk, or otherwise taken into the body to sustain life, provide energy, promote growth, etc. Examples of food delivery vehicles or food products of interest include, but are not limited to: baby or infant formula, baby food (e.g., pureed food suitable for infant or toddler consumption), chips, cookies, breads, spreads, creams, yogurts, liquid drinks, chocolate containing products, candies, ice creams, cereals, coffees, pureed food products, etc.

Also of interest as oral formulations are food supplements. Where the oral formulation is provided as a food supplement, the food supplement may further include one or more of a sweetener, a stabilizer, a flavoring or a colorant, etc. An oral formulation according to the present disclosure may be provided in the form of sugar-coated tablets or lozenges, pills, gelatin capsules, or syrups. Oral formulations may be provided as a bulk sample, e.g., a container having multiple doses in powder form that can be measured out by a subject, or in unit dose form, e.g., a pill, pouch, single use container, and the like.

Tablets may contain the active ingredient in admixture with non-toxic physiologically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period.

Formulations for oral use may also be presented as hard gelatin capsules wherein the 2′-FL is mixed with an inert solid diluent, for example, calcium carbonate, calcium phosphate or kaolin, or as soft gelatin capsules wherein the composition is mixed with water or an oil medium.

Aqueous suspensions contain 2′-FL in admixture with excipients suitable for the manufacture of aqueous suspensions. Such excipients may include suspending agents, for example sodium carboxymethyl-cellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The physiologically acceptable compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The 2′-FL can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The composition can be utilized in aerosol formulation to be administered via inhalation. The composition can also be formulated into pressurized acceptable propellants such as dichlorodifluoromethane, propane, or nitrogen.

The compositions can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Also of interest are topical compositions, e.g., where the composition is combined with a topical delivery vehicle component. The topical delivery vehicle component of the delivery compositions of the invention may vary, as desired, where the particular ingredients of a given delivery vehicle component will depend, at least in part, on the nature of the particular composition. Delivery compositions of interest include liquid formulations, such as lotions (liquids containing insoluble material in the form of a suspension or emulsion, intended for external application, including spray lotions) and aqueous solutions, semi-solid formulations, such as gels (colloids in which the disperse phase has combined with the dispersion medium to produce a semisolid material, such as a jelly), creams (soft solids or thick liquids) and ointments (soft, unctuous preparations), and solid formulations, such as topical patches. As such, delivery vehicle components of interest include, but are not limited to: emulsions of the oil-in-water (O/W) and the water in-oil (W/O) type, milk preparations, lotions, creams, ointments, gels, serum, powders, masks, packs, sprays, aerosols or sticks.

The amount of the 2′-FL composition that may be combined with the carrier materials to produce a single dosage form will vary depending upon the host treated and the particular mode of administration. For example, a unit formulation intended for the oral administration of humans may contain from 0.5 mg to 5 g of composition compounded with an appropriate and convenient amount of carrier material which may vary from about 0.1 to about 95 percent of the total composition, or e.g. about 0.1 to about 10 percent, about 0.1 to about 4 weight percent, or about 5 to about 95 percent of the total composition. It will be understood, however, that the specific dose level for any particular patient will depend upon a variety of factors including the age, body weight, general health, sex, diet, time of administration, route of administration, rate of excretion, drug combination and the severity of the particular disease undergoing therapy. As such, unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier. As described above “unit dosage forms,” include physically discrete units suitable as unitary dosages for human and animal subjects, each unit containing a predetermined quantity of the composition calculated in an amount sufficient to produce the desired effect in association with a pharmaceutically acceptable diluent, carrier or vehicle.

### Clinical Use of 2′-FL for Treatment of ASD

Any of the 2′-FL compounds [free (unconjugaged) oligosaccharide, glycoconjugate form, or combinations, e.g., mixtures of oligosaccharides] and/or compositions comprising the same, e.g., those described herein, are administered to a subject in need thereof, e.g., those described herein, for treating ASD. For example, the subject is a human patient at risk of developing ASD or the subject is a human patient having ASD, e.g., diagnosed as having neurobehavioural disorder(s) associated with ASD. For example, the subject is a human ASD patient who has undergone or is on an ASD-related therapy (e.g., a pharmaceutical, physiological, and/or physical therapy). For example, the subject is a human ASD patient who has been diagnosed with active ASD or one who is in remission of the ASD and is receiving an ASD-related therapy (e.g., a pharmaceutical, physiological, and/or physical therapy).

Any of the 2′-FL compounds and/or compositions comprising the same, e.g., those described herein, can be administered to a subject of any age who is in need of ASD treatment. In some examples, a subject to be administered a 2′-FL compound and/or composition described herein can be a child, for example, a subject who is 18 years old or younger, e.g., 6 months-18 years old, inclusive. In some embodiments, the subject may be a child at the age of 11 or over, e.g., 11-18 years old, inclusive. In some embodiments, the subject may be a child at the age of 5-10. In some embodiments, the subject may be a child under the age of 5, e.g., 6 months to 4 years old, inclusive. In some embodiments, a subject to be administered a 2′-FL compound and/or composition described herein can be an adult who is over the age of 18, such as 19-80 years old, inclusive. In some embodiments, an adult subject is at the age of 19-25. In some embodiments, an adult subject to be administered a 2′-FL compound and/or composition described herein may be above 25 (e.g., 25-80 years old, inclusive). In some embodiments, an adult subject to be administered a 2′-FL compound and/or composition described herein may be an elderly who is over the age of 65, such as 66-80 years old. In some embodiments, subjects to be administered a 2′-FL compound and/or composition described herein may be at the age of 11 to 25.

The term “treating” or “treatment” as used herein refers to application or administration of a 2′-FL compound, as a monotherapy or as a combined treatment (e.g., as an adjunct to an immune system suppression and/or anti-inflammatory therapy) to a subject, who has ASD, a symptom of ASD, or a predisposition toward ASD, with the purpose to cure, heal, alleviate, relieve, alter, remedy, ameliorate, improve, or affect the disease, the symptoms of the disease, or the predisposition toward the disease. The term “treating” or “treatment” also includes application or administration of a 2′-FL compound, as a monotherapy or as a combined treatment (e.g., as an adjunct to an immune system suppression and/or anti-inflammatory therapy) to a subject who is in remission of ASD, with the purpose to maintain the remission and thus alleviate, reduce, prevent, or delay the relapse occurrence.

In some embodiments, the treatment is prophylactic. The term “prophylactic” refers to application or administration of a 2′-FL compound, as a monotherapy or as a combined treatment (e.g., as an adjunct to an immune system suppression and/or anti-inflammatory therapy) to a subject who is at risk for ASD that prevents the occurrence, or delays the onset, of ASD.

In some embodiments, the treatment is therapeutic. The term “therapeutic” refers to application or administration of a 2′-FL compound (e.g., ones described herein, either as a purified oligosaccharide, mixture or in glycoconjugate form as described herein), as a monotherapy or as a combined treatment (e.g., as an adjunct to an immune system suppression and/or anti-inflammatory therapy) to a subject, who has ASD or a symptom of ASD that improves at least one or more symptoms associated with ASD, as described herein, including e.g., reduced gastrointestinal symptoms, microbial dysbiosis, mitochondrial dysfunction, and/or thermal dysregulation, and/or reduced frequency of symptom relapse.

For example, a treatment is therapeutic when application or administration of a 2′-FL compound (e.g., ones described herein, either as a purified oligosaccharide or in glycoconjugate form as described herein), as a monotherapy or as a combined treatment (e.g., as an adjunct to an immune system suppression and/or anti-inflammatory therapy) alleviates or reduces the risk of ASD symptom relapse. As used herein, the term “relapse” refers to the occurrence or worsening of at least one or more symptoms associated with ASD.

An “effective amount” refers to an amount of a 2′-FL compound (e.g., ones as described herein), that alone, or together with further doses, produces the desired response, e.g., elimination or alleviation of symptoms, prevention or reduction the risk of symptom relapse in ASD, a reduction in at least one symptom of ASD described herein. The desired response is to inhibit the progression or relapse of the symptoms of the disease. This may involve only slowing the progression of the disease temporarily, although it may involve halting the progression of the disease permanently. In some instances, this may involve only delaying the relapse of the disease temporarily, although it may involve preventing the relapse of the disease permanently. This can be monitored by routine methods. The desired response to treatment of the disease also can be delaying the onset or even preventing the onset of the disease. Such amounts will depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

For example, an effective amount of a 2′-FL compound when administered to a subject in need thereof results in, e.g., by increasing the abundance of intestinal microbes that produce short-chain fatty acids by at least about 10% or more, including, e.g., at least about 20%, at least about 30%, at least about 40%, at least about 50%, at least about 60%, at least about 70%, at least about 80%, at least about 90% or more, as compared to the abundance of short-chain fatty acid-producing intestinal microbes without administration of the 2′-FL compound. Examples of intestinal microbes that produce short-chain fatty acids (e.g., butyrate) include, but are not limited to Bifidobacteria, Bacteroides, and/or Parabacteroides. In some embodiments, an effective amount of a 2′-FL compound (e.g., ones as described herein) when administered to a subject in need thereof results in increasing the abundance of intestinal Bifidobacteria by at least about 10% or more, including, e.g., at least about 20%, at least about 30%, at least about 40%, at least about 50%, at least about 60%, at least about 70%, at least about 80%, at least about 90% or more, as compared to the abundance of intestinal Bifidobacteria without administration of the 2′-FL compound. Such therapeutic features can be determined by measuring the abundance of fecal microbes (e.g., Bifidobacteria, Bacteroides, and/or Parabacteroides).

In some embodiments, an effective amount of a 2′-FL compound[(e.g., ones as described herein, either as a free (unconjugated) oligosaccharide or in glycoconjugate form, or in a mixture of oligosaccharides as described herein] when administered to a subject in need thereof results in, e.g., by increasing microbial butyrate production by at least about 10% or more, including, e.g., at least about 20%, at least about 30%, at least about 40%, at least about 50%, at least about 60%, at least about 70%, at least about 80%, at least about 90% or more, as compared to the microbial butyrate production without administration of the 2′-FL compound. Such therapeutic feature can be determined by measuring the abundance of fecal short-chain fatty acid including, e.g., butyrate.

In some embodiments, an effective amount of a 2′-FL compound when administered to a subject in need thereof results in, e.g., by decreasing intestinal inflammation by at least about 10% or more, including, e.g., at least about 20%, at least about 30%, at least about 40%, at least about 50%, at least about 60%, at least about 70%, at least about 80%, at least about 90% or more, as compared to the intestinal inflammation without administration of the 2′-FL compound. Such therapeutic features can be determined by diagnosis and measuring methods as described herein.

In some embodiments, an effective amount of a 2′-FL compound when administered to a subject in need thereof results in, e.g., by decreasing intestinal inflammation by at least about 10% or more, including, e.g., at least about 20%, at least about 30%, at least about 40%, at least about 50%, at least about 60%, at least about 70%, at least about 80%, at least about 90% or more, as compared to the intestinal inflammation without administration of the 2′-FL compound. Such therapeutic features can be determined by diagnosis and measuring methods as described herein. An exemplary effective amount of a 2′-FL compound for use in the methods described herein can be equivalent to at least 0.5 mg/day of 2′-fucosyllactose, at least 1 mg/day of 2′-fucosyllactose, at least 2 mg/day of 2′-fucosyllactose, at least 3 mg/day of 2′-fucosyllactose, at least 4 mg/day of 2′-fucosyllactose, at least 5 mg/day of 2′-fucosyllactose, at least 6 mg/day of 2′-fucosyllactose, at least 7 mg/day of 2′-fucosyllactose, at least 8 mg/day of 2′-fucosyllactose, at least 9 mg/day of 2′-fucosyllactose, at least 10 mg/day of 2′-fucosyllactose, at least 11 mg/day of 2′-fucosyllactose, at least 12 mg/day of 2′-fucosyllactose, at least 13 mg/day of 2′-fucosyllactose, at least 14 mg/day of 2′-fucosyllactose, at least 15 mg/day of 2′-fucosyllactose, at least 16 mg/day of 2′-fucosyllactose, at least 17 mg/day of 2′-fucosyllactose, at least 18 mg/day of 2′-fucosyllactose, at least 19 mg/day of 2′-fucosyllactose, or at least 20 mg/day of 2′-fucosyllactose. In some embodiments, an effective amount of a 2′-FL compound (e.g., ones as described herein) for use in the methods described herein can be equivalent to no more than 20 mg/day of 2′-fucosyllactose, no more than 15 mg/day of 2′-fucosyllactose, no more than 10 mg/day of 2′-fucosyllactose, no more than 9 mg/day of 2′-fucosyllactose, no more than 8 mg/day of 2′-fucosyllactose, no more than 7 mg/day of 2′-fucosyllactose, no more than 6 mg/day of 2′-fucosyllactose, no more than 5 mg/day of 2′-fucosyllactose, no more than 4 mg/day of 2′-fucosyllactose, no more than 3 mg/day of 2′-fucosyllactose, or no more than 2 mg/day of 2′-fucosyllactose. Combinations of the above-recited ranges are also included. For example, in some embodiments, an effective amount of a 2′-FL compound (e.g., ones as described herein) for use in the methods described herein can be equivalent to 0.5 mg/day to 20 mg/day of 2′-fucosyllactose, equivalent to 1 mg/day to 20 mg/day of 2′-fucosyllactose, equivalent to 1 mg/day to 15 mg/day of 2′-fucosyllactose, equivalent to 1 mg/day to 10 mg/day of 2′-fucosyllactose, equivalent to 1 mg/day to 8 mg/day of 2′-fucosyllactose, or equivalent to 1 mg/day to 5 mg/day of 2′-fucosyllactose. In some embodiments where a subject in need of the treatment is at the age of 11-25, an effective amount of a 2′-FL compound for use in the methods described herein can be equivalent to 1 mg/day to 20 mg/day of 2′-fucosyllactose, equivalent to 1 mg/day to 15 mg/day of 2′-fucosyllactose, equivalent to 1 mg/day to 10 mg/day of 2′-fucosyllactose, equivalent to 1 mg/day to 8 mg/day of 2′-fucosyllactose, or equivalent to 1 mg/day to 5 mg/day of 2′-fucosyllactose.

The daily effective amount of a 2′-FL compound can be administered in a single daily dose, or divided into multiple doses (e.g., 2-4 doses) for administration at given time intervals during the day. In some embodiments, the daily effective amount of a 2′-FL compound (e.g., ones as described herein) can be administered as a single daily dose in the morning, e.g., alone or in combination with food or beverages. Administration of a 2′-FL compound (e.g., ones as described herein) at any other times during the day is also suitable.

A 2′-FL compound can be administered to a subject in need thereof as a single oligosaccharide for treatment of ASD or in combination with at least one additional oligosaccharides (e.g., ones described herein). In some embodiments, a 2′-FL compound (e.g., ones as described herein) is administered to a subject in need thereof as a single oligosaccharide, i.e., the subject is given a 2′-FL compound (e.g., ones as described herein) as the only oligosaccharide, which is not co-used with other oligosaccharides (e.g., ones described herein). In other embodiments, a 2′-FL compound (e.g., ones as described herein) is co-administered with at least one different oligosaccharide. By “co-administered” or “in combination with” is meant that a subject is provided with a 2′-FL compound (e.g., ones as described herein) with a different oligosaccharide during the course of treatment, such as concurrently, consecutively, intermittently, or in other regimens.

A 2′-FL compound can be administered as an adjunct to an ASD-treating agent, e.g., one being taken by a human ASD patient. As used herein, the term “adjunct” refers to a first agent being provided as a supplement to a second agent. The first agent can be administered prior to, concurrently with, or after administration of the second agent. In some embodiments, administration of a 2′-FL compound as an adjuvant to an ASD-treating agent or therapy can provide a synergistic effect on treatment of ASD, including, e.g., alleviating or reducing the risk of relapse in ASD. For example, the therapeutic effect is synergistic when the average duration of remission achieved by the combination of a 2′-FL compound (e.g., ones described herein) and an optional ASD-treating agent or therapy is significantly greater than the additive effect ensuing from individual treatment with the same doses of a 2′-FL compound (e.g., ones described herein) and an ASD-treating agent or therapy. In some embodiments, the synergistic therapeutic effect increases the average duration of remission by at least 20%, 30%, 40%, 50%, 60%, 70%, 80%, 90% or more.

When a 2′-FL compound is co-administered with a second agent (e.g., other oligosaccharides as described herein and/or other ASD-treating agents or therapies), it may be formulated together with the second agent in a single composition, which may be in any suitable form as described herein (e.g., powder or tablets for oral administration). Alternatively, the 2′-FL compound (e.g., ones described herein) and the second agent (e.g., other oligosaccharides as described herein and/or other ASD-treating agents or therapies) may be formulated separately.

Administration of ASD treatment described herein may be accomplished by any method known in the art (see, e.g., Harrison's Principle of Internal Medicine, McGraw Hill Inc., 18th ed., 2011). For combined treatment, each agent can be administered via the same route or different routes. Administration may be local or systemic. Administration may be, for example, parenteral (e.g., intravenous, intraperitoneal, subcutaneous, intra-arterial or intradermal), or oral. Compositions for different routes of administration are well known in the art (see, e.g., Remington: The Science and Practice of Pharmacy, Pharmaceutical Press, 22^{nd} ed., 2012). The compositions may also be formulated as modified release dosage forms, including delayed-, extended-, prolonged-, sustained-, pulsed-, controlled-, accelerated- and fast-, targeted-, programmed-release, and gastric retention dosage forms. These dosage forms can be prepared according to conventional methods and techniques known to those skilled in the art. Dosage will depend the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Dosage can be determined by the skilled artisan. In some embodiments, a 2′-FL compound (e.g., ones described herein) and/or a second agent (e.g., other oligosaccharide(s) as described herein and/or other ASD-treating agents or therapies) can be administered orally. Oral administration also includes buccal, lingual, and sublingual administration. In some embodiments, compositions comprising a 2′-FL compound (e.g., ones described herein) may be provided in solid, semisolid, or liquid composition (e.g., pharmaceutical composition or dietary supplement) for oral administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the compositions may contain one or more pharmaceutically acceptable or edible carriers or excipients, including, but not limited to, binders, fillers, diluents, disintegrants, wetting agents, lubricants, glidants, coloring agents, dye-migration inhibitors, sweetening agents, and flavoring agents.

In some embodiments, a 2′-FL compound (e.g., ones described herein) can be administered by injection (e.g., parenterally such as intravenously or intraperitoneally).

Preparations for parenteral administration include sterile aqueous or non-aqueous solutions, suspensions, and emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain one or more of a preserving agent, a wetting agent, an emulsifying agent and a dispersing agent. The dosage forms may be sterilized by, for example, filtration of the composition, by irradiating the composition, or by heating the composition. They can also be manufactured using sterile water, or some other sterile injectable medium, prior to use. Additional information regarding administration of 2′FL is known in the art, e.g., as described in Morrow et al., PCT Publication No. WO2018/187792 A1 (International Application No. PCT/US2018/026631); hereby incorporated by reference.

In some embodiments, the method further comprises taking actions other than or in addition to an ASD treatment described herein. In some embodiments, the method further comprises monitoring development of an ASD symptom of a subject who is at risk for ASD, or monitoring the effectiveness of the treatment. The monitoring may comprise a physical examination, endoscopy, and/or stool sample examination, e.g., for assessing intestinal inflammation and/or intestinal microbiota. If the subject is not responsive to an administered dose of a 2′-FL compound, a physician can increase the dose of the 2′-FL compound, e.g., based on the medical and/or physical condition of the subject, provided that the increased dose does not cause significant gastrointestinal symptoms such as bloating, abdominal pain, nausea, loose stools, and/or gassiness.

### Methods of Therapy

The composition is suitable for an infant, a toddler, a child, or an adult with ASD. For example, the methods are useful to shift the microbiota of the subject to a more favorable status using 2′-FL, alone or in combination with other human milk oligosaccharides, thereby resulting in an improvement in the disorder.

### Evaluation of ASD-Associated Sociality Disorders

A rodent model was developed to understand “sociality” in non-human animals (e.g., laboratory rodents) and especially how social affiliations are established, formed and maintained. This model is highly relevant to “translational” problems, such as animal models of autism, because every syndrome along the Autism Spectrum Disorders (ASDs) and many other psychiatric diseases involve disturbance of social behavior, attachment, and social responsivity. Such disorders are now viewed as developmental disorders, so the studies of the development of sociality pertain to the etiology/foundation of such types of disease.

The studies address “group behavior” displayed by littermates of rat and mouse pups, revealing how such family groups (including the mother) function as a behavioral and physiological unit. Moreover, while the individuals function as a group, the studies also evaluate individuals in a group and this enables observation and analysis of how group behavior and individual behavior co-exist interdependently and how each one shapes the other. This dual perspective led to a computational model that explains the formation and dynamics of group behavior, based on rules of individual behavior, revelations on how sex differences within the group leads to different social experiences, and how neurohormones such as oxytocin are stimulated by specific interactions with the mother and how manipulations of oxytocin in the offspring brain, even just some of the pups can alter the entire family dynamic in lawful, quantifiable ways.

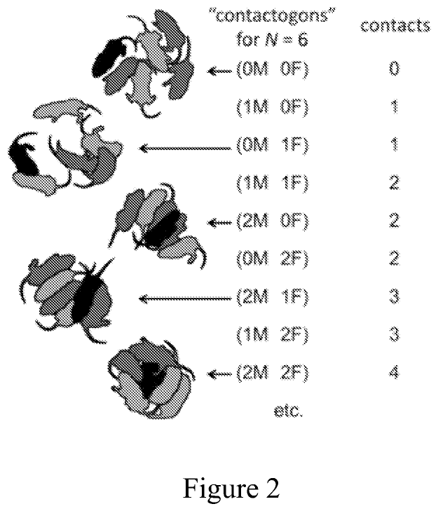

FIG. 2 illustrates one of a variety of related methods we use to capture the complexities of simultaneous individual and group behavior. This example shows four, separate examples of group organization depicting the contact patterns, positions, sexes (female=light gray; male=dark gray) and identification of the “target” individual (black) in this snapshot. Automatically captured frames like these are assembled into a time series and analyzed to track the individuals' contacts as well as changes in the “fission” and “fusion” among group members.

The area subtended by the group is also measured, which provides another objective measure of closeness, or cohesion among individuals. The place of the mother (also called the “dam”) is also observed, and the social dynamics of the family in relation to the dam or without her involvement is observed. Along with these different modes of description, a suite of tools, defining “aggregons”, “contactagons”, family “size” and other units of analysis, were developed.

A primary sensory and physiological factor that attracts pups to one another and to the mother is body heat. Heat transfer (conduction) between bodies is a vital part of how pups are attracted to the mother and littermates. The resulting conductive heat exchange is the basis of learned affiliations in which odors of the mother and siblings become the cues for social affiliation and preferences. This is the basis of their social bonding and a key to the formation of that aspect of their sociality.

During early development of rats, mice and humans (this is fundamental to all mammals) there is special importance to body heat, both physiologically and socially. Close contact (skin to skin) increases the “social hormone” oxytocin in the brains of rat pups. Blocking the transmission of oxytocin in the infant brain during the social interactions that establish social bonds (the odor learning mentioned earlier) blocks the learning.

All mammalian infants possess a host of specializations involving the production and recognition of heat. There is a special organ, unique to mammals, called brown adipose tissue (BAT) that is the source of an infant's heat-producing (thermogenic) capability. Mutant mice that have been genetically-engineered for the display of diminished social responses show deficits in heat-production and body temperature regulation. Similarly, animals bred for deficits in BAT physiology (BAT—knockouts) are reported to show social deficits. Such data support the premise that there is a developmental foundation for normal and abnormal social development that pervasively involves the production and exchange of physiological heat.

The heat produced by BAT thermogenesis is noteworthy because it arises from an identified alteration in the metabolic pathways within the mitochondria of the brown fat cells. Many overlapping clues between such mitochondrial function and the expression of normal and abnormal sociality. The active role of 2′-FL in regulating mitochondrial function, augmenting the production of body heat, the findings of lower body temperatures in dysbiotic pups and their corresponding deficits in sociality, and the restoration of body temperature regulation and social responses with 2′-FL, represent a strong foundation for the therapeutic use of oligosaccharides alone and in combination with other substances for remediation of gut microbiota, thermoregulatory function, and the development of normal, adaptive sociality in young mammals.

### Example 1: Interpersonal Sociality is Rescued by Administration of 2′-FL to ASD Subjects

ASD is a neurobehavioral disorder in which affected individuals have persistent deficits in social communication and interaction (symptom of ASD). To address the impact of 2′-FL administration on social communication and interaction, a mouse model for ASD was designed and experiments conducted to study ASD-associated neurobehavioral disorders such as interpersonal sociality. A depiction of the system used for scoring contact behavior was adapted from known method, e.g., that described by Harshaw, C. et al. (2014). “Sex Differences in Thermogenesis Structure Behavior and Contact within Huddles of Infant Mice.” Plos One, 9(1), 1-15 and Sokoloff G, Blumberg M S. Competition and cooperation among huddling infant rats. Dev Psychobiol. 2001 September; 39(2):65-75. doi: 10.1002/dev.1030, Erratum in: Dev Psychobiol 2001 November; 39(3):229.

The question to be addressed was whether providing 2′-FL rescues the normal sociality of mouse pups whose social interaction was perturbed. Perturbation of behavior was achieved by antibiotic treatment of their mothers using a method previously developed and validated. Sociality is measured by objective measurement of “aggregons” which means the (a) huddling of dams and pups or (b) pups only in groups.

The mouse experiment involved 22 mother-pup litters including a control group (n=7 litters with 28 pups), an antibiotic treated group (7 litters with 28 pups), and a 2′-FL treated group (8 litters with 32 pups).

Mothers were treated with an antibiotic cocktail, starting from gestational day 12; delivery was on day 20. Antibiotic use was continued for mothers postpartum. Treatment of 2′-FL was given to mothers and pups through their water intake. Sociality was measured at night, as mice are nocturnal animals, using the mean number of aggregons (social groupings). Aggregons could consist of individual mice only if they were not huddled with other mice, or they could be a group defined as either one or more pups with the dam, or two or more pups without the dam. The outcome of the experiment was measured on postpartum day 7. A higher number of aggregons signifies less sociality—more time alone or time in groups with fewer individuals—whereas, a lower number of aggregons signifies more sociality—more time spent by individuals with others, including in larger groupings.

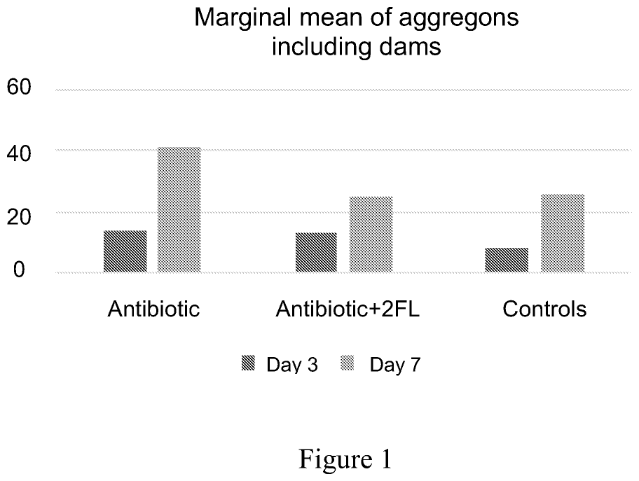

On night 7, the antibiotic-treated group (no 2′-FL), had a marginal mean of 41 aggregons when considering only those aggregons that included dams, significantly (p<0.05) higher than the marginal mean of 26 aggregons for the controls (FIG. 1). The 2′-FL treated group followed the pattern of the controls with a marginal mean of 25 aggregons. This pattern in the aggregons by experimental group was also observed for pup-only aggregons (p<0.05). This signifies that sociality was significantly less for antibiotic-treated mice (dams and their offspring) than controls, but that normal sociality was rescued by administration of 2′-FL.

These data indicate that administration of 2′-FL is useful to reduce a symptom of ASD in a subject, thereby providing significant clinical benefit to individuals characterized as comprising ASD.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

1. A method for reducing a symptom of autism spectrum disorder (ASD) in a subject, comprising administering to said subject a composition comprising purified 2′-fucosyllactose, wherein said subject comprises a symptom of ASD.

2. The method of claim 2, wherein said subject is a human.

3. The method of claim 1, wherein said subject is less than 7 years of age.

4. The method of claim 1, wherein said subject is less than 3 months of age.

5. The method of claim 1, wherein said subject is an adult.

6. The method of claim 1, wherein said symptom comprises gastrointestinal dysfunction, microbial disbiosis, mitochondrial dysfunction, thermal dysregulation, immune dysregulation, a social deficit, or a cognition deficit.

7. The method of claim 1, wherein the composition comprises a food product.

8. The method of claim 11, wherein the food product comprises a pureed food product.

9. The method of claim 11, wherein the food product comprises infant formula.

10. The method of claim 1, wherein said subject is administered 5-8 grams of 2′FL per day.

11. The method of claim 1, wherein interpersonal sociality is improved following administration of said 2′FL.

12. A composition comprising 2′-FL for use in a treatment to reduce reducing a symptom of autism spectrum disorder in a subject.
